SEQUENCE LISTING (9 sequences)

SEQ ID NO: 1 - DNA, 1460 bp, Artificial Sequence
  Synthetic
ggccgcccaccatgggggaggtttgaagtgtgcgcctgatataatcatacacctaaaagcaccacttgctgattgtgaagggactatgtcgtttatgacgggacgttacgctggccgatggtttgaatttggacgctgtggtagaatgttatatggacgtaaaggttggcatattgaaaatcgtcttcacaggcaaacttctagacgtgtgacccaccggtaaaacgacaagcgtggcgcgtcgattgcgctttgaacgtcgtttgttggactccagatgaacctcaaaatcaaagcggtgattgacgaaaatcaaatgacagcccgcaaaatttcatcagccttcggatcggattctcagaatctgattgtccctgctggctacatttatgaaatttcgtacattttggcagaaatgtcccaataccatagcactgccgcctgagctcacccgagcaatgcatactgggtacctcgcccatctcgccctctttccaagcccagtgctgttgtaaatagccaaagggctcagtaacaatggccaaactgacatccgctgttcctgtgttgacagcaagagatgttgcaggtgcagtggagttttggacagatagactggggtttagcagggactttgtggaggacgattttgcaggagtggtgagggatgatgtgacactgtttatctcagcagtgcaggatcaagtggtgcccgataatacactggcatgggtttgggtgagaggattggatgaactgtatgcagagtggtctgaagtggtgagcaccaactttagggatgcaagcggacctgcaatgacagagattggagaacaaccttggggaagggagtttgcattgagagatcctgcagggaattgcgtgcactttgttgcagaagaacaggactgagcatagcatcagcctgtggcagggttgtggtagggctgagtggcagggttaaaggggttgcctaccccacccctactctcatgacaccagcaacagcagcagctcatgcagtactcaaatcactgatgtcaatggtgtgacacatttggttaaggctgctttttaaagtgctgctttgggggcagtgactgtgcagagcttggagcgtatccccatgtaatcagaaccgacgagagttcggggcaacctttcatcttcacattttttgtgatcagctacagagtctgaaatcaaatagaggctgccatctaaacgcaggagtcacaacgaaggcgaaaactccaattgctgtactcaatgcactaagtgattgttcaatggataaatacactatgctcaattcatgccagcagagctgctccttccagccagctacaatggctttttccacgccttttgaagtatgaatgttcagcttgctgtgcttgatgcatcaccataaacacaattctacaacatttcatgccaacaacagtacgggctttcgg

SEQ ID NO: 2 - DNA, 3752 bp, Artificial Sequence
  Synthetic
ggccgcccaccatgggggaggtttgaagtgtgcgcctgatataatcatacacctaaaagcaccacttgctgattgtgaagggactatgtcgtttatgacgggacgttacgctggccgatggtttgaatttggacgctgtggtagaatgttatatggacgtaaaggttggcatattgaaaatcgtcttcacaggcaaacttctagacgtgtgacccaccggtaaaacgacaagcgtggcgcgtcgattgcgctttgaacgtcgtttgttggactccagatgaacctcaaaatcaaagcggtgattgacgaaaatcaaatgacagcccgcaaaatttcatcagccttcggatcggattctcagaatctgattgtccctgctggctacatttatgaaatttcgtacattttggcagaaatgtcccaataccatagcactgccgcctgagctcacccgagcaatgcatactgggtacctcgcccatctcgccctctttccaagcccagtgctgttgtaaatagccaaagggctcagtaacaatggccaaactgacatccgctgttcctgtgttgacagcaagagatgttgcaggtgcagtggagttttgtgagttctgagaagctgattgttgtttaacttctttgaaagctttatcgaagattctgcaagcgatgaacattgcttgtcaagaccgagagctgcatgcccacttgacatccagctttgaacggctcttcatgtttgatttgtttctgattgtagggacagatagactggggtttagcagggactttgtggaggacgattttgcaggagtggtgagggatgatgtgacactgtttatctcagcagtgcaggatcaagtgagtgcagcgtcagctgtggcagttgttggctttcgtctcagtcagtagtttgctgggattgattatggagggcacagttgcaattttgagttgcacgttgcgacaagcgtgttgacaaagcgtggtcaagccggccagtcttgccggtggcgggtggcttggtctaacttccgctctacagcaatcgttttgttcatggttacggggctggcgtgccagaaagtcctggtcagccaccctcgcttcaaagccgtagcccaacaactttgcgaatatgttcgatttgcaggtggtgcccgataatacactggcatgggtttgggtgagaggtacagctctgcgtgcaacaggttgcaagatgcagcgcaggtcttccctggtcaaacgatgtatgcagagttgagaggcacttgagctgggtgaatggcgtgggctcgtaggtagtgtgcagggcaggaagggcagccaattttggagttgtggtccggtgtcgttgcttcgagccttattaggactcttgctcatcaaagcgttagttgtgaataagttgatctgaaaggatgttatgtacagcaagcagcagcagttaagagtctggggagtagctgcacagggcgaggtgtcaagatgggaagggtcctgcctccttatgtgtttttccctgtaggggaggaagcctcttatgggcaatggttgggcatattttccagccagcccttctttctataggggccagggtgggcccagctcgtcttggcttccaccaccaggagagtgagggcattgaagggccataaatagtcctcccatctacgtgcaccagagggtgtcgtctaggctgtgcatgccacgaggggaaggagccaagaatgagtgtatgggttgttttcatgtttaggctgggataaaactgttttcaattgcgcctgccgggtgaaaaccacagcagcatcagcaagcttggagaaggccagcccgcccagcacaggctcacgttcccactcaggcggtcagtcgggcgggggtgtgagtcaggcaggcgagggtgtctgtgcctgacatcagcacctctgcttagccactgcagcccctggagcagggtagggcgtcatttgcagcaatcacctgctgcctcacacgtcgcagcttggaatttcaacgaccatcagcgctggggttgttgagggatcatagcagattttggtgcagcctggttgtcatgctctttgtggaatggcctctatgttcgagcaattcgttggatgttgaggtgcttggggacagagagtcgaatgatgggccagggtcaaacatgcgagcgtttggctgagtcagcggtttttgctggtcactttttcttttgtttcttatttaggtttgatggatgtgttttgtgctgctgccctgaagctgcagcagcgtgtctgccctgcgctactgcgggcaccaaggctatgtgctggtgcactcggctgcgctgcacctgtgcacctcgcactccgtccagcctccatgcagcacacgtactcacggtgtcctcctgacctgtcgtacgctattccaaacttgctcttttgctgccgctgctctcgtacacaattgctgttgattatcgatatctaatcgagcgcctgctgactgaactccgcaggtttggatgaactgtatgcagagtggtctgaagtggtgagcaccaactttaggtgggtgggctctgaaggaggaggagggagcgggtgattaaacagggcctgcatgaagaggagcaggggctgcatggacagcagggggaaggtgcagaagggagggtcaagcggggttcaggtggctgtgggtttctgcacgagcagtgaaagaagctgtatccttccacctgctttcactggcgaaaggttgaaaacaggatgtcgcagctggaaagatgttgcgctgtcaagtgcaagccatggttgagggtatgcctgtgtgcatgtgcttcttaaagttactcctgttctatggttctgggtgcttgttgtttgtggtgcagggatgcaagcggacctgcaatgacagagattggagaacaaccttggggaagggagtttgcattgagagatcctgcaggtgagggggcatgtaagcaatggcaggcaattcaagaacgaatcattgctgcaaatgctgggatggtatgcagctgaggtatctattgccttgtattttgtctcgcattgcatcggtggtgcgttctgtggcctgaggcacagttcttgctgtttgataagggttcgactgagttgtcgtgtgtgctgtgctgcaggcaattgcgtgcactttgttgcagaagaacaggactgagcatagcatcagcctgtggcagggttgtggtagggctgagtggcagggttaaaggggttgcctaccccacccctactctcatgacaccagcaacagcagcagctcatgcagtactcaaatcactgatgtcaatggtgtgacacatttggttaaggctgctttttaaagtgctgctttgggggcagtgactgtgcagagcttggagcgtatccccatgtaatcagaaccgacgagagttcggggcaacctttcatcttcacattttttgtgatcagctacagagtctgaaatcaaatagaggctgccatctaaacgcaggagtcacaacgaaggcgaaaactccaattgctgtactcaatgcactaagtgattgttcaatggataaatacactatgctcaattcatgccagcagagctgctccttccagccagctacaatggctttttccacgccttttgaagtatgaatgttcagcttgctgtgcttgatgcatcaccataaacacaattctacaacatttcatgccaacaacagtacgggctttcgg

SEQ ID NO: 3 - RNA, 103 nt, Artificial Sequence
  Synthetic
gggacauggugcgcaaggacguuuuagagcuagaaauagcaaguuaaaauaaggcuaguccguuaucaacuugaaaaaguggcaccgagucggugcuuuuuuu

SEQ ID NO: 4 - DNA, 120 bp, Artificial Sequence
  Synthetic
taatacgactcactatagggacatggtgcgcaaggacgttttagagctagaaatagcaagttaaaataaggctagtccgttatcaacttgaaaaagtggcaccgagtcggtgcttttttt

SEQ ID NO: 5 - DNA, 120 bp, Artificial Sequence
  Synthetic
aaaaaaagcaccgactcggtgccactttttcaagttgataacggactagccttattttaacttgctatttctagctctaaaacgtccttgcgcaccatgtccctatagtgagtcgtatta

SEQ ID NO: 6 - DNA, 32 bp, Artificial Sequence
  Synthetic
tgcgacatgcagcttactaacctgctcgacat

SEQ ID NO: 7 - DNA, 24 bp, Artificial Sequence
  Synthetic
cccccagcctcacatccgcctcaa

SEQ ID NO: 8 - DNA, 21 bp, Artificial Sequence
  Synthetic
acccaaacccatgccagtgta

SEQ ID NO: 9 - DNA, 25 bp, Artificial Sequence
  Synthetic
actgtatgcagagtggtctgaagtg

(19) United States
(12) Patent Application Publication
(10) Pub. No.: US 2017/0130238 A1
(43) Pub. Date: May 11, 2017

(54) DEVICE AND METHODS FOR BIOLISTIC TRANSFORMATION

(71) Applicant: SYNTHETIC GENOMICS, INC., La Jolla, CA (US)

(72) Inventors: Amanda R. Edwards, Carmichael, CA (US); John H. Verruto, San Diego, CA (US)

(21) Appl. No.: 15/343,064

(22) Filed: Nov. 3, 2016

Related U.S. Application Data
(60) Provisional application No. 62/250,397, filed on Nov. 3, 2015.

(51) Int. Cl. C12N 15/82

(52) U.S. Cl. CPC C12N 15/8207

(57) ABSTRACT

The present invention provides an innovate method and device for depositing coated particles to prepare sample cartridges for use in a gene gun. It allows for scalability of sample cartridge preparation, as well as the ability to prepare multiple different samples simultaneously thereby reducing the time required to prepare different samples and consequently the time required to perform a biological assay, such as a transformation as

### CROSS REFERENCE TO RELATED APPLICATION(S)

This application claims benefit of priority under 35 U.S.C. 119(e) to U.S. Ser. No. 62/250,397, filed Nov. 3, 2015, the entire contents of which is incorporated herein by reference in its entirety.

### INCORPORATION OF SEQUENCE LISTING

The material in the accompanying sequence listing is hereby incorporated by reference into this application. The accompanying sequence listing text file, name SGII1940_1_Sequence_Listing, was created on Nov. 3, 2016, and is 10 kb. The file can be assessed using Microsoft Word on a computer that uses Windows OS.

### FIELD OF INVENTION

The present invention relates generally to the field of particle delivery into cells, and more particularly to a method and device for depositing coated particles in the preparation of sample cartridges for use in a gene gun system.

### BACKGROUND

Following the discovery of the genetic materials and rapid developments in genetics, scientists now can skillfully practice genetic engineering technology, for example, introducing foreign genes to affect the physiology of a cell or even an organism. Gene transfer has been widely applied in scientific studies and in improvements of agricultural products, where heterologous genetic materials, such as DNA molecules, are transferred to host cells in order to change the biological characteristics and morphology of the cells.

In recent years, gene transformation using a physical approach has been successfully applied to cells, microorganisms, and plant and animal tissues. The physical approach, utilizing a particle gun (also referred to as a gene gun), is to accelerate metal particles carrying the biological materials (e.g., DNA molecules) into cells for gene transformation or gene transfer. This approach is applicable to the research and development in fields such as, for example, plant biology, agricultural improvement, mammalian somatic cell biology, gene therapy, and the recently developed DNA vaccination.

The gene gun system uses gold or tungsten particles coated with DNA or RNA within a sample cartridge and a gas to accelerate the cartridge based on the high pressure shock wave principle. When a preset high pressure is reached in the pressurized chamber, the sample cartridge having DNA-coated particles is accelerated by a resulting shock wave into a stopping screen. The DNA-coated particles continue to accelerate to enter the target tissue due to the inertia effect.

One particular particle gun system is the Helios® Gene Gun System sold by Bio-Rad® (Hercules, Calif.). The Helios® Gene Gun System includes of all of the components needed to prepare DNA-coated microcarriers, coat the DNA-microcarrier suspension onto the inner surface of the Gold-Coat™ tubing, cut the tubing into cartridges which are used in the Helios® Gene Gun, and finally propel the microcarriers and their associated DNA into cells.

Prior to transfer to a cell, the plasmid DNA must be attached to the gold particles. This is typically accomplished by precipitation of the DNA from solution in the presence of gold microcarriers and the polycation spermidine by the addition of CaCl₂. The particles are then washed extensively with ethanol to remove the water and resuspended in ethanol. Using a Tubing Prep Station™ device (shown in FIG. 1) the DNA microcarrier solution is coated onto the inner wall of Gold-Coat™ (Tefzel™) tubing and dried. The tubing is then cut into 0.5 inch length cartridges using the Tubing Cutter™. These cartridges, when inserted into the cartridge holder of the Helios® Gene Gun are the source of the DNA which enters the target cells by the helium discharge.

Preparation of bullets for the Helios® Gene Gun System entails applying and drying DNA-coated gold on the inside of Tefzel™ (ethylene tetrafluoroethylene) tubing using the Tubing Prep Station™ in accordance to the manufacturer's protocol (found in the Helios® Gene Gun System Instruction Manual, M1652411 available at bio-rad.com/en-us/product/helios-gene-gun-system?tab=Documents). As per the manufacturer's protocol, after pulling the DNA-coated gold suspended in ethanol into a thirty-inch length of Tefzel™ tubing, the tubing is inserted into the Tubing Prep Station™, and the gold settled out of suspension adhering to the inside of the Tefzel™ tubing. After removing the ethanol, the Tefzel™ tubing, which is fixed at both ends to the Prep Station device, is rotated continuously by the Tubing Prep Station™ while blowing nitrogen gas through the Tefzel™ tubing to dry the gold. After the drying process is complete, the tubing is cut into 0.5-inch lengths to fit into the Helios® Gene Gun. These half-inch tubing segments including the dried DNA-coated gold particles adhered to the inner surface of the tubing become the cartridges of the gene gun, sometimes referred to herein as “bullets”.

This method of preparation has several disadvantages. First, using the Tubing Prep Station™, each set of bullets requires at least thirty minutes to prepare. Though the first steps of precipitation of DNA onto the gold, washing of the gold to remove water, and resuspension of the DNA-coated gold in ethanol could accommodate multiple samples simultaneously, only one sample may be coated and dried onto the Tefzel™ (Bio-Rad's® Gold-Coat™) tubing at a time. For each sample, the Tefzel™ tubing must be pre-dried for at least 15 minutes prior to gold application, then the gold has to settle out of suspension for five minutes before the removal of the ethanol, and then at least ten minutes is required for drying the gold inside the Tefzel™ tubing. If it is desired to transform ten different DNA samples plus a positive and negative control in a single experiment, a full day is needed to prepare the bullet set needed for the single transformation experiment.

Another problem with the Tubing Prep Station™ is the difficulty in scale-down of the number of bullets when fewer than forty bullets per sample are desired. The Tubing Prep Station™ is designed to make 40 bullets per DNA sample, i.e., from a single DNA-particle prep, with the amount of DNA and gold microparticles for a single “bullet prep” recommended by the manufacturer of the Tubing Prep Station™ being 50 ug and 25 mg, respectively. Reduction of the number of bullets is not feasible using the manufacturer's protocol because the apparatus is too long to be able to manipulate a shorter length of Tefzel™ tubing.

Another problem with the Tubing Prep Station™ is the potential DNA cross-contamination between sets of bullets. When using the Tubing Prep Station™, one end of the Tefzel™ tubing is dipped into the gold suspension while the other end is attached to a syringe to apply and hold suction. After pulling the gold suspension into the Tefzel™ tubing, the same end that was dipped into the gold is inserted into the Tubing Prep Station™ at which point any residual DNA and/or gold present on the outside of the Tefzel™ tubing can be left on the upstream O-ring and motor region.

Accordingly, a need exists for an improved method and device for sample cartridge preparation which addresses the disadvantages of conventional methods and devices.

### SUMMARY OF THE INVENTION

The present invention provides an innovative method and device for depositing coated particles in the preparation of sample cartridges for use in a gene gun. The method does not require continuous rotation of the tubing used to form gene gun sample cartridges. The present invention allows for scalability of sample cartridge preparation, as well as the ability to prepare multiple different samples simultaneously thereby reducing the time required to prepare different samples and consequently the time required to perform a biological assay, such as a transformation assay. Additionally, the present invention addresses potential problems of sample cross-contamination.

Accordingly, in one aspect, the present invention provides a method for simultaneously depositing particles on the interior surfaces of a plurality of separate pieces of tubing. The method includes:

a) preparing a suspension of particles coated with a biological substance in an evaporable liquid;

b) introducing the particle suspension into each of a plurality of separate pieces of tubing; and

c) simultaneously passing a gas through each piece of tubing of the multiple pieces of tubing via a manifold to dry the particles onto the interior surface of each of the plurality of separate pieces of tubing. The manifold includes an elongated lumen defining a central fluid flow pathway, and a plurality of fluid connector ports disposed along the lumen. In exemplary embodiments, multiple separate pieces of tubing are each in fluid connection with an individual fluid connector port whereby the gas is allowed to simultaneously flow from the lumen of the manifold into each piece of tubing, thereby simultaneously depositing particles on the interior surface of each of the separate pieces of tubing.

The particle suspension is introduced into each separate piece of tubing by any feasible means, for example, by coupling the syringe filled with the particle solution to an end of a piece of tubing prior to connecting the separate piece of tubing to a manifold fluid connector port, and drawing the suspension of particles into the piece of tubing using the syringe from the end opposite to the end connected to the syringe.

The gas for drying the coated particles in the pieces of tubing is passed through the pieces of tubing. The time period for gas to be passed through the pieces of tubing can in some examples be for about 5-20 minutes to dry the particles. For example the particles may be dried in less than about 20, 15, 14, 13, 12, 11, 10, 9, 8, 7, 6, or 5 minutes.

In further embodiments, the method further includes cutting each piece of tubing having dried particles along its length to produce a plurality of individual tubing segments of about 0.5 inches in length which may then be inserted into a cartridge holder and used in a gene gun. The particles within a piece of tubing or cartridge produced therefrom include particles coated with a biological molecule deposited onto the interior surface via the method of the invention. In various embodiments, the particles may be non-uniformly deposited on the interior surface of the piece of tubing.

In yet another aspect, the present invention provides an apparatus for performing the method of the invention. The apparatus includes a manifold, the manifold being fluidly coupled to one or more pieces of tubing of the invention. The manifold includes an elongated lumen defining a central fluid flow pathway, and a plurality of fluid connector ports disposed along the lumen, each piece of tubing being in fluid connection with a fluid connector port of the manifold. A piece of tubing in fluid connection with a fluid connector port of the manifold can be free at the tubing end opposite the end in fluid connection with the fluid connector port. For example, during operation of the device, each piece of tubing in fluid connection with a fluid connector port of the manifold can be free at the tubing end opposite the end in fluid connection with the fluid connector port.

In various embodiments, during or after operation of the device, one or more of the pieces of tubing (or cartridges generated therefrom) can include particles coated with two or more different biomolecules, for example, at least one DNA molecule and at least one RNA molecule. As nonlimiting examples, an RNA molecule can be a crispr RNA or a tracr RNA, and can be a guide RNA, which may or may not be a chimeric (or “single”) guide RNA. A DNA molecule coating particles can be a DNA molecule that comprises a selectable marker or detectable marker (“reporter gene”). In various embodiments, a prepartion of particles in a piece of tubing can include at least one DNA molecule and at least two RNA molecules, or at least two DNA molecules and at least two RNA molecules. In various embodiments, an RNA molecule used to coat a particle can be, without limitation, a funcional RNA such as but not limited to transactivating RNA (tracrRNA), cripsr RNA (crRNA), single guide RNA, chimeric guide RNA, RNAi construct, shRNA, siRNA, antisense RNA sequence, ribozyme, or microRNA. An RNA molecules can also be a translatable RNA molecule that encodes a polypeptide.

A further aspect of the invention is a method for coating particles for bombardment of cells with at least one RNA molecule and at least one DNA molecule. In a first embodiment, the method includes: preparing a slurry of metal particles in a solution of spermidine; adding a DNA molecule to the slurry of metal particles; adding calcium chloride to the slurry of metal particles and DNA; pelleting the metal particles; resuspending the particles in aqueous solution; adding RNA to the particles in aqueous solution; and alcohol precipitating the RNA and metal particles. In another embodiment, the method includes: preparing a slurry of metal particles in aqueous solution with at least one DNA molecule and at least one RNA molecule; and alcohol precipitating the RNA and metal particles. The particles can be, for example, gold or tungsten. Alcohol precipitation can be precipitation with ethanol or isopropanol and can also include addition of a salt, such as but not limited to ammonium acetate or sodium acetate. The method can include coating particles with at least one DNA molecule and at least two RNA molecules. The method can include coating particles with at least two DNA molecule and at least two RNA molecules. For example, the method can include coating particles with at least one DNA molecule that includes a selectable marker cassette and at least one crispr RNA or guide RNA.

In some embodiments the method includes coating particles for bombardment of cells with at least one DNA molecule and at least two RNA molecules. For example, the method can include coating particles for bombardment of cells with at least one DNA molecule and at least two guide RNA molecules. The DNA molecule can optionally include a selectable marker cassette. In additional embodiments, the method can include coating particles for bombardment of cells with at least two DNA molecule and at least two guide RNA molecules. The at least two DNA molecules can optionally each include a different selectable marker cassette conferring resistance to different antibiotics.

Further included are preparations of particules for bombardment of cells in which the particles are coated with at least two RNA molecule and at least one DNA molecule. Further included are are preparations of particules for bombardment of cells in which the particles are coated with at least two RNA molecules and at least two DNA molecules. In any of the above embodiments, an RNA molecule can be a crRNA or guide RNA, and may be a chimeric guide RNA.

In a further aspect, the present invention provides a method of delivering at least one biological molecule to a cell. The method includes:

a) providing a piece of tubing prepared according to the method of the invention, wherein the lumen of the piece of tubing includes deposited particles coated with at least one biological molecule; and

b) contacting the cell with the coated particles deposited within the piece of tubing via a gene gun device, thereby delivering the one or more biological molecules to the cell.

In still another aspect, the invention provides a method of delivering at least one nucleic acid molecule to a cell. The method includes:

a) providing a piece of tubing prepared according to the method of the invention, wherein the lumen of the piece of tubing includes deposited particles coated with at least one nucleic acid molecule; and

b) contacting the cell with the nucleic acid molecule-coated particles deposited within the piece of tubing via a gene gun device, thereby delivering the one or more nucleic acid molecule to the cell.

### BRIEF DESCRIPTION OF THE DRAWINGS

FIG. 1 is a simplified diagram illustrating a prior art drying device used for preparation of gene gun sample cartridges from the Helios® Gene Gun System Instruction Manual, M1652411 (available at bio-rad.com/en-us/product/helios-gene-gun-system?tab=Documents): 1) syringe adapter tubing for attachment to Tefzel™ tubing for loading particle sample; 2) support bar for rotating tubing; 3) clamp; 4) syringe sleave; 5) tubing rotation support; 6) syringe adapter tubing; 7) female luer fitting; 8) motor housing; 9) syringe adapter tubing; 10) male luer fittings; 11) position switch; 12) flow meter; 13) valve regulating nitrogen fow; 14) nitrogen hose; 15) spur gear.

FIG. 2 is an image of one embodiment of a manifold drier device of the present invention: 1) manifold; 2) tubing for feeding nitrogen gas to manifold; 3) site for insertion of Tefzel™ tubing into original Bio-Rad® Prep Station™; 4) LPM regulator (flow meter); 5) Tefzel™ tubing pieces; 6) Leur locks.

FIG. 3 is a schematic diagram of the cpSRP54 gene locus and the primers for diagnosing a Cas9-mediated insertion of a selectable marker donor fragment into the targeted locus.

FIGS. 4A-4B. (A) provides gels showing PCR products resulting from PCR with primers designed to diagnose the insertion of the selectable marker cassette into the cpSRP54 locus of Parachlorella WT-01185 versus the wild type cpSRP54 locus. The leftmost gel shows only the wild type locus is amplified when the particle preparation does not include a guide RNA, whereas the gel on the right shows that coating the particles with the RNA guide together with the donor DNA that include the ZeoR cassette provides three colonies of twelve tested having PCR products indicative of integration of the donor DNA; (B) the results of sequentially coating particles with donor DNA and guide RNA provides six of twelve colonies tested have PCR products indicative of integration of the donor DNA ZeoR cassette, and the rightmost gel shows the controls A: line having random integration of pSGE-6543 and B: Cas9-expressing parental strain GE-15699 both show the PCR product of the wild type cpSRP54 locus, and C: no PCR template shows no amplification product is made.

### DETAILED DESCRIPTION OF THE INVENTION

The present invention provides an innovative method and manifold drier device for preparation of sample cartridges for use in a gene gun.

Before the present compositions and methods are further described, it is to be understood that this invention is not limited to the particular systems, methods, and experimental conditions described, as such systems, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth. All references cited herein are incorporated by reference in their entireties.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein a “gene gun” is a device designed for particle bombardment of biological cells, organisms, or tissue, that includes a chamber for positioning of particles, typically coated with a biological substance such as one or more nucleic acid molecules, proteins, or peptides, where the chamber is in fluid communication with a barrel or channel open at the end opposite from the chamber, through which the particles are propelled when the gun is activated. Typically the end of the chamber opposite from the channel or barrel is connected to a source of gas, such as nitrogen or helium, whose flow into the chamber propels the particles though the barrel or channel and out of the gun. Gene guns available commercially include the Helios® Gene Gun from Bio-Rad® (Hercules, Calif.) and the Auragen Accell® gene gun. The methods and compositions presented herein can be used with these devices for transfer of biological substances into cells or can be used with other devices designed according to the same principles for propelling particles coated with a biological substance into cells (see, for example, U.S. Pat. Nos. 6,194,389; 7,449,200, 7,449,449; 7,901,711; 8,137,697; 8,449,915, and U.S. Patent App. Pub. No. 2004/0033589, all of which are incorporated herein by reference in their entireties).

As used herein a “biological substance” is a substance that includes at least one biomolecule, that may be, for example, a carbohydrate, protein, peptide, or nucleic acid molecule, such as a DNA or RNA molecule. (For example, as used herein, biological molecules include, but are not limited to proteins, peptides and fragments thereof (whether naturally occurring, chemically synthesized or recombinantly produced), as well as nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like.) A biological substance can include more than one biological molecule, and/or can include more than one type of biological molecule, and for example, can include a molecular complex (e.g., a protein-RNA complex). In some embodiments provided herein, a biological substance comprises at least one RNA molecule and at least one DNA molecule. A different biological substance differs in composition from a referenced biological substance by at least one particular molecule, for example, a specific RNA molecule or DNA molecule.

A “particle” or “carrier particle” as used herein is a particle intended for use as a projectile that is propelled into a cell of interest and preferably is coated with a biological substance such as at least one nucleic acid molecule or polypeptide. A particle may be any suitable material, such as, for example, ceramic or metal, or can even comprise a biodegradable material such as chitosan, and is preferably metal, such as tungsten or gold. A particle can range in diameter from about 0.2 μm to about 2.0 μm, and is preferably from about 0.3 μm to about 1.8 μm in diameter, for example, from about 0.4 μm to about 1.7 μm in diameter, or from about 0.5 μm to about 1.6 um in diameter.

“An evaporable liquid” can be any liquid that can evaporate under the provided conditions, including water or an aqueous buffer, an alcohol such as ethanol, methanol, or isopropanol, or an organic solvent such as acetone or chloroform, or mixtures of any thereof. Preferably the evaporable liquid is an alcohol, such as ethanol.

“RNA-guided nuclease” is used herein to refer generically to enzymes of CRISPR systems in which the referred to nuclease hydrolyzes DNA in a site-specific manner, where the targeted site is determined by an RNA molecule that interacts with the nuclease. Examples of RNA-guided nucleases include but are not limited to Cas1, Cas1B, Cas2, Cas3, Cas4, Cas5, Cas6, Cas7, Cas8, Cas9, Cas10, Cbf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, C2c1, C2c2, homologs thereof, and modified versions thereof.

A “CRISPR system” or “CRISPR-cas system” refers to a Cas protein, such as but not limited to a Cas9 protein or a variant thereof, or a nucleic acid molecule encoding a Cas protein, along with one or more RNAs required for targeting and/or altering a genetic locus. For example, a CRISPR-cas system can include a Cas protein or a nucleic acid molecule encoding a Cas protein and at least one tracrRNA (“trans-activating CRISPR RNA”) or gene encoding a tracr RNA and at least one crRNA or “CRISPR RNA” or gene encoding a crRNA, in which the crRNA comprises sequences homologous to a target nucleic acid sequence. The crRNA, which may also be referred to as a guide RNA, may further include a “tracr mate” sequence that is able to hybridize with the tracrRNA. Alternatively, a CRISPR system can include a cas protein (or a gene or transcript encoding a cas protein) and a gene or transcript that includes both the tracrRNA and crRNA sequences. A single RNA molecule that includes both a tracr sequence and a cr (target homologous) sequence is referred to herein as a “chimeric guide RNA” (or simply a “guide RNA”). A crRNA or guide RNA can further include a tracr-mate sequence (encompassing a “direct repeat” and/or a tracrRNA-processed partial direct repeat as in an endogenous CRISPR system). In some embodiments, one or more elements of a CRISPR system is derived from a type I, type II, or type III CRISPR system. CRISPR-cas systems and their use in genome editing are disclosed in Jinek et al. (2012) Science 337:816-821; Brouns (2012) Science 337:808; Gaj et al. (2013) Trends in Biotechnol. 31:397-405; Hsu et al. (2013) Cell 157:1262-1278; Mali et al. (2013) Science 339:823-826; Qi et al. (2013) Cell 152:1173-1183; Walsh & Hochedlinger (2013) Proc Natl Acad Sci 110:155414-155515; Sander & Joung (2014) Nature Biotechnology; Sternberg et al. (2014) Nature 507:63-67; U.S. Pat. App. Pub. No. 2014/0068797; U.S. Pat. No. 8,697,359; U.S. Pat. App. Pub. No. 2014/0170753; U.S. Pat. App. Pub. No. 2014/0179006; U.S. Patent No. 20140179770; U.S. Pat. App. Pub. No. 2014/0186843; and U.S. Pat. App. Pub. No. US 2015/0045546; all of which are incorporated by reference in their entireties.

In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. A sequence or template that may be used for recombination into the targeted locus comprising the target sequences is referred to as an “editing template” or “editing sequence”, “donor sequence” or “donor DNA”. In aspects of the invention, an exogenous template polynucleotide may be referred to as a donor DNA molecule.

The term “selectable marker” or “selectable marker gene” as used herein includes any gene that confers a phenotype on a cell in which it is expressed to facilitate the selection of cells that are transfected or transformed with a nucleic acid construct of the invention. The term may also be used to refer to gene products that effectuate said phenotypes. Nonlimiting examples of selectable markers include: 1) genes conferring resistance to antibiotics such as amikacin (aphA6), ampicillin (amp^{R}), blasticidin (bls, bsr, bsd), bleomicin or phleomycin (ZEOCIN™) (ble), chloramphenicol (cat), emetine (RBS14p or cry1-1), erythromycin (ermE), G418 (GENETICIN™) (neo), gentamycin (aac3 or aacC4), hygromycin B (aphIV, hph, hpt), kanamycin (nptII), methotrexate (DHFR mtx^{R}), penicillin and other β-lactams (β-lactamases), streptomycin or spectinomycin (aadA, spec/strep), and tetracycline (tetA, tetM, tetQ); 2) genes conferring tolerance to herbicides such as aminotriazole, amitrole, andrimid, aryloxyphenoxy propionates, atrazines, bipyridyliums, bromoxynil, cyclohexandione oximes dalapon, dicamba, diclfop, dichlorophenyl dimethyl urea (DCMU), difunone, diketonitriles, diuron, fluridone, glufosinate, glyphosate, halogenated hydrobenzonitriles, haloxyfop, 4-hydroxypyridines, imidazolinones, isoxasflutole, isoxazoles, isoxazolidinones, miroamide B, p-nitrodiphenylethers, norflurazon, oxadiazoles, m-phenoxybenzamides, N-phenyl imides, pinoxadin, protoporphyrionogen oxidase inhibitors, pyridazinones, pyrazolinates, sulfonylureas, 1,2,4-triazol pyrimidine, triketones, or urea; acetyl CoA carboxylase (ACCase); acetohydroxy acid synthase (ahas); acetolactate synthase (als, csr1-1, csr1-2, imr1, imr2), aminoglycoside phosphotransferase (apt), anthranilate synthase, bromoxynil nitrilase (bxn), cytochrome P450-NADH-cytochrome P450 oxidoreductase, dalapon dehalogenase (dehal), dihydropteroate synthase (sul), class I 5-enolpyruvylshikimate-3-phosphate synthase (EPSPS), class II EPSPS (aroA), non-class I/II EPSPS, glutathione reductase, glyphosate acetyltransferase (gat), glyphosate oxidoreductase (gox), hydroxyphenylpyruvate dehydrogenase, hydroxy-phenylpyruvate dioxygenase (hppd), isoprenyl pyrophosphate isomerase, lycopene cyclase, phosphinothricin acetyl transferase (pat, bar), phytoene desaturase (crtI), prenyl transferase, protoporphyrin oxidase, the psbA photosystem II polypeptide (psbA), and SMM esterase (SulE) superoxide dismutase (sod); 3) genes that may be used in auxotrophic strains or to confer other metabolic effects, such as arg7, his3, hisD, hisG, lysA, manA, metE, nit1, trpB, ura3, xylA, a dihydrofolate reductase gene, a mannose-6-phosphate isomerase gene, a nitrate reductase gene, or an ornithine decarboxylase gene; a negative selection factor such as thymidine kinase; or toxin resistance factors such as a 2-deoxyglucose resistance gene.

A “detectable marker”, “detectable marker gene”, or “reporter gene” is a gene encoding a protein that is detectable or has an activity that produces a detectable product. A reporter gene can encode a visual marker or enzyme that produces a detectable signal, such as cat, lacZ, uidA, xylE, an alkaline phosphatase gene, an α-amylase gene, an α-galactosidase gene, a β-glucuronidase gene, a β-lactamase gene, a horseradish peroxidase gene, a luciferin/luciferase gene, an R-locus gene, a tyrosinase gene, or a gene encoding a fluorescent protein, including but not limited to a blue, cyan, green, red, or yellow fluorescent protein, a photoconvertible, photoswitchable, or optical highlighter fluorescent protein, or any of variant thereof, including, without limitation, codon-optimized, rapidly folding, monomeric, increased stability, and enhanced fluorescence variants.

An expression cassette or simply, “cassette” is used herein to refer to a gene operably linked to one or more regulatory elements to drive the expression of the gene. Typically an expression cassette includes a gene (for example, a selectable marker gene) operably linked to a promoter and, optionally, a terminator sequence. An expression cassette may also comprise sequences that enable, mediate, or enhance translation of the nucleotide sequence. The coding region usually codes for a protein of interest but may also code for a functional RNA of interest, for example antisense RNA or a non-translated RNA, in the sense or antisense direction. An expression cassette may be assembled entirely extracellularly (e.g., by recombinant cloning techniques). The expression of the nucleotide sequence in the expression cassette may be under the control of a constitutive promoter or of an inducible promoter which initiates transcription only when the host cell is exposed to some particular external stimulus.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Examples of expression vectors known in the art include cosmids, plasmids and viruses (e.g., retroviruses, lentiviruses, adenoviruses, and adeno-associated viruses) that incorporate the recombinant polynucleotide.

The present invention provides a reproducible method for producing sample cartridges for use in a gas-driven gene gun system. In particular, small dense carrier particles are reversibly coated onto a concave inner surface of a piece of tubing which serves as a sample cartridge. The carrier particles are themselves reversibly coated with a biological substance, for example, at least one biological molecule such as a nucleic acid molecule or a protein. During particle acceleration and delivery, a gas stream passing over the carrier particles releases the same from the inner surface of the sample cartridge, and carries the particles to a target cell, tissue, or organism.

In one aspect, the invention provides a method for simultaneously depositing particles on the interior surfaces of a plurality of separate pieces of tubing. The method includes: a) preparing a suspension of particles coated with a biological substance in an evaporable liquid; b) introducing the particle suspension into each separate piece of tubing; and c) simultaneously passing a gas through each piece of tubing via a manifold to dry the particles onto the interior surfaces of a plurality of tubing pieces. The manifold includes an elongated lumen defining a central fluid flow pathway, and a plurality of fluid connector ports disposed along the lumen. In embodiments, each piece of tubing is in fluid connection with a fluid connector port whereby the gas is allowed to simultaneously flow from the lumen into each piece of tubing, thereby simultaneously depositing particles on the interior surface of each of the pieces of tubing.

The method in various examples does not include continuous rotation of the pieces of tubing during drying of the particle suspension within the piece of tubing, for example, does not include continuous rotation of the tubing during the time period when gas is passed through the tubing.

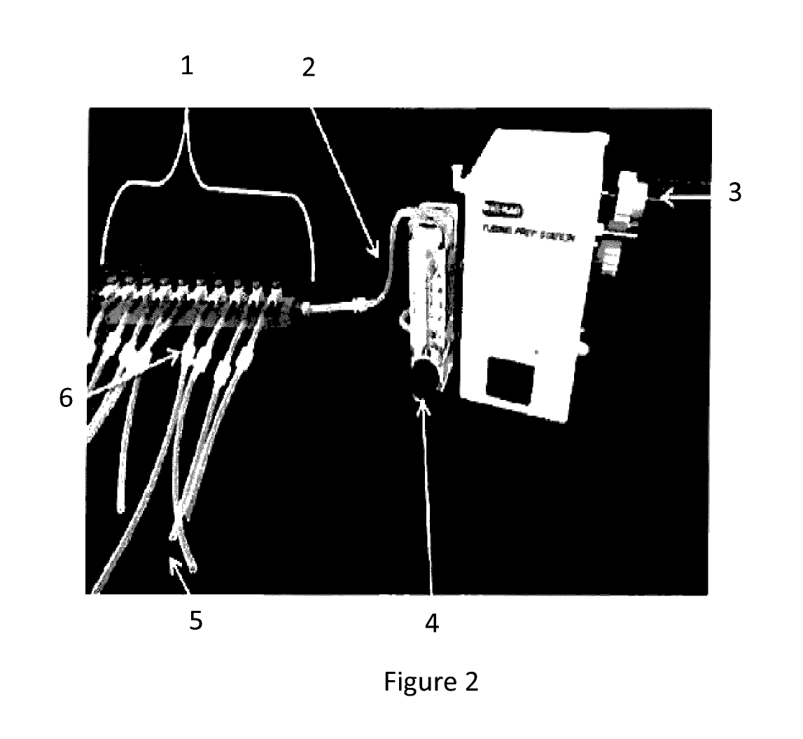

The present invention utilizes an apparatus having a manifold, the manifold being fluidly coupled to one or more pieces of tubing of the invention. Referring to FIG. 2, the manifold is shown attached to and in fluid communication with a gas supply for supplying gas (e.g., air or nitrogen) to the manifold. The manifold is a structure that can be made of any feasible material, for example, plastic, that includes an elongated lumen defining a central fluid flow pathway, and a plurality of fluid connector ports disposed along the lumen, each piece of tubing being in fluid connection with a fluid connector port of the manifold. The manifold allows a plurality of individual pieces of tubing to be attached to the manifold such that gas may be passed through the lumen of each piece of tubing allowing for particles in each piece of tubing to be deposited simultaneously by drying. Each piece of tubing may include particles comprising the same or a different biological substance (where a different biological substance can include at least one different specific molecule, e.g., a different DNA construct or guide RNA) such that a multitude of samples of different types may be prepared simultaneously thereby drastically reducing sample prep time.

The manifold apparatus may include one or more valves in order to provide for controlled delivery of gas into the manifold. Additionally, gas flow into each piece of tubing may be controlled by individual valves fluidly coupled to each fluid connector port of the manifold. As such, gas may be supplied to certain pieces of tubing and not others at the discretion of the user.

Operation of the components of the deposition apparatus may be controlled manually, or alternatively, operation of one or more components is governed electronically, for example, by a commercially available programmable controller and electrically actuated valves. As will be appreciated by the skilled artisan upon reading the instant specification, a microprocessor unit can be used to direct operation of the programmable controller, allowing for fully automated operation of the deposition apparatus. For example, an appropriate set of drying gas flow rates can be entered into the microprocessor, which then controls operation of the controller and valves over an entire deposition procedure. Alternatively, the microprocessor allows for semiautomatic operation of the deposition apparatus, such as where one or several cycles of the deposition procedure are under the control of the microprocessor, while parameters of other operations are controlled manually.

The method of the invention requires preparation of coated particles for depositing onto interior surfaces of individual pieces of tubing of the manifold device provided herein. A suspension of particles coated with a biological substance in an evaporable liquid is prepared and introduced into each separate piece of tubing before being dried via attachment to the manifold.

Biological molecules can be coated onto carrier particles using a variety of techniques known in the art. A biological substance may be used interchangeably herein with the term “one or more biological molecules”. Dense materials, such as gold and tungsten, are preferred as a carrier particle in order to provide particles that can be readily accelerated toward a target over a short distance, wherein the coated particles are still sufficiently small in size relative to the cells into which they are to be delivered, for example, less than about 2 μm in diameter, preferably about 1.6 μm in diameter or less, and in various examples may be less than or equal to about 1 μm in diameter. In some examples, the particles may be less than or equal to about 0.5 μm in diameter, for example, less than or equal to about 0.4 μm in diameter. Any method known in the art can be used to prepare the coated particles, however a preferred method for coating DNA onto gold particles is described herein. One of ordinary skill in the art will appreciate from the following description the importance of determining, within acceptable tolerance limits, the amount of biological substance per particle and the number of particles per sample cartridge.

Gold or tungsten particles may be utilized for coating with a nucleic acid molecule, such as RNA or DNA. References herein to “beads” or “particles” are intended to include, without limitation, both spherical and amorphous particles of appropriate size and density. DNA is one biological molecule that may be coated onto particles. RNA is another biological molecule that may be coated onto particles. However, other substances including, but not limited to, proteinaceous materials can also be coated onto particles using the following techniques. In this regard, conditions for depositing other biological substances or for using non-gold particles can vary from the method stated in ways that are understood in the art. As provided herein, a biological substance can comprise more than one type of biomolecule, e.g., at least one DNA molecule and at least one RNA molecule can be coated on the same preparation of particles.

To prepare the coated particles, a desired amount of particles, with may be tungsten or gold particles, is selected. The amount of particles to be used can be roughly determined by multiplying the desired amount of particles per delivery by the number of sample cartridges being prepared, e.g., the number of cartridges produced from one piece of tubing. A suitable amount of particles per delivery is typically on the order of about 0.25 to 0.50 mg of gold particles per delivery, although acceptable amounts can be higher or lower. For a 7 inch piece of tubing, for example, a suitable amount of gold particles may be from about 5-12 mg or from about 8-10 mg. Of course, one of the advantages of the manifold device and method is that the piece of tubing used to generate bullets for a single coated particle preparation is adjustable at the discretion of the user, such that the amount of gold particles used for a bullet prep may be significantly more than 12 mg or less than 5 mg.

In various examples, protein or a nucleic acid molecule such as DNA is precipitated onto gold particles using methods well known in the art. The amount of DNA used to coat approximately 8-10 mg of gold particles can be from about 10-25 μg or from about 15-20 μg for the exemplified 7 inch long piece of tubing. Again, the amount of DNA can be significantly more or less depending on the length of the piece of tubing. DNA or RNA used to coat particles can be circular or linear, single-stranded, double-stranded, or partially single-stranded and partially double-stranded. Coating of gold particles with DNA is accomplished by precipitation of the DNA from solution in the presence of gold particles and the polycation spermidine by the addition of calcium chloride (CaCl₂). In order to obtain the most uniform coating results, the volume of DNA should not exceed the volume of spermidine, but smaller volumes may be used. Accordingly, it may be necessary to adjust either the concentration of DNA or the volume of spermidine added initially to the gold particles. Calcium chloride is added to result in precipitation of DNA-coated gold particles. The particles are then washed extensively with ethanol to remove the water. The coated particles containing known amounts of both DNA and gold, are resuspended in an evaporable liquid, preferably 100% ethanol, optionally containing an appropriate amount of an additive that provides a slight, temporary adhesive effect sufficient for joining the coated particles to the sample cartridge. One such suitable adhesive is polyvinyl pyrrolidone (PVP), which may be present, for example, at concentrations of from about 0.01 to 0.1 mg/ml. Methods and compositions for coating particles may vary and are not limiting to the invention.

In other examples, the particles are coated with one or more RNA molecules, for example, by alcohol precipitation with the particles in a solution that can include a salt such as ammonium acetate. In the RNA bullet preparation method described in the Helios® manual, the measured gold powder is suspended first in aqueous RNA Ammonium acetate (one tenth volume) and two volumes of isopropanol are added, and the sample is incubated at −20° C. for 1 hour to precipitate the RNA in the presence of the gold. After the 1 hour incubation, the sample is pelleted, washed, and applied to Tefzel™ tubing in the same way as DNA coated bullets.

Provided herein are methods of coating particles for bombardment of cells with both DNA and RNA, for example, at least one DNA molecule and at least one RNA molecule. A first method includes coating particles with DNA according to methods provided hereinabove, e.g., preparing a slurry of metal particles in a solution of spermidine, adding a DNA molecule to the slurry of metal particles in a spermidine solution, adding calcium chloride to the slurry of metal particles and DNA, pelleting the metal particles; and resuspending the particles in aqueous solution to provide DNA-coated particles; and then binding RNA to the DNA-coated particles by adding RNA to the DNA-coated particles in aqueous solution and alcohol precipitating the RNA and DNA-coated metal particles to produce RNA and DNA coated particles. The particles can be suspended in a slurry, for example with aqueous solution or alcohol.

In another embodiment of the method, both DNA and RNA are ethanol precipitated with the metal particles to simultaneously coat the particles with RNA and DNA. The method includes: preparing a slurry of metal particles in aqueous solution with at least one DNA molecule and at least one RNA molecule and alcohol precipitating the RNA, DNA, and metal particles to provide particles coated with RNA and DNA. The particles can be suspended in a slurry, for example with aqueous solution or alcohol.

The particles used in the methods can be, for example, gold or tungsten. Alcohol precipitation can be precipitation with ethanol or isopropanol and can also include addition of a salt, such as but not limited to ammonium acetate or sodium acetate. The method can include coating particles with at least one DNA molecule and at least two RNA molecules. The method can include coating particles with at least two DNA molecule and at least two RNA molecules. For example, the methods can include coating particles with at least one DNA molecule that includes a selectable marker cassette and at least one crispr RNA or guide RNA.

In some embodiments the method includes coating particles for bombardment of cells with at least one DNA molecule and at least two RNA molecules. For example, the method can include coating particles for bombardment of cells with at least one DNA molecule and at least two guide RNA molecules. The DNA molecule can optionally include a selectable marker cassette. In additional embodiments, the method can include coating particles for bombardment of cells with at least two DNA molecule and at least two guide RNA molecules. The at least two DNA molecules can optionally each include a different selectable marker cassette conferring resistance to different antibiotics.

For preparation of gene gun cartridges, a suspension of coated particles (particles coated with RNA, DNA, or both RNA and DNA molecules) is then introduced into a piece of tubing of desired length, which can be, in nonlimiting examples, from about 3 inches to about 20 inches in length. The tube may be pre-dried by attachment to the manifold device and passing a gas through the tubing for about 1, 2, 3, 4, 5, 6, 7, 8, 9, 10 minutes or longer before being loaded with solution. Since the invention allows for scalability, any number of tubing pieces (such as at least 2, 3, 4, 5, 6, 7, 8, 9, 10, 11, 12 or more separate pieces of tubing) may be utilized, each of any desired length (such as less than about 12, 10, 9, 8, 7, 6, 5 or 4 inches in length). Lengths of tubing of between about 5-7 inches are preferable and provide a sufficient number of cartridges to perform an assay without wasting materials. Additionally, while a number of polymeric materials are known in the art and are suitable for use in the present invention as a tube, in embodiments, the tube is composed of ethylene tetrafluoroethylene (ETFE), which is sold under the tradename Tefzel™. Alternatively the tubing can be composed of any plastic resistant to the chemicals used, including but are not limited to, for example, polytetrafluoroethylene (PTFE), fluorinated ethylene propylene (FEP), and perfluoroalkoxy copolymer resin (PFA). While the dimensions of the tubing may vary and are not limiting, the tubing diameter will advantageously fit the cartridge holder of the gene gun, and for the commonly-used and commercially available Helios® Gene Gun, can be tubing of approximately 3.175 mm in outer diameter and 2.36 mm in inner diameter.

In one embodiment, the suspension of coated particles is introduced into a piece of tubing by attachment of the tube to a syringe via a first intermediate tubing segment fluidly connecting the tube to the syringe. The open end of the piece of tubing is dipped into the solution and drawn into the tube via suction. While maintaining a connection between the syringe and the piece of tubing, the piece of tubing that includes the suspension of coated particles is placed on a flat surface for about 1, 2, 3, 4, 5, 6, 7, 8, 9 or 10 minutes, preferably at least 5 minutes, to allow particles to settle out of solution and adhere to the interior surface of the piece of tubing. Since the percentage of the gold that adheres to the piece of tubing is influenced by the settling time, it is important to keep the settling time consistent for every sample. Ideally, a timer is utilized and the step of drawing the gold into the piece of tubing is staggered to allow for adequate handling time for each sample in subsequent steps.

After the particles have been allowed to settle, ethanol is removed from the tube by application of pressure to the syringe to gently push the ethanol from the tube. In some embodiments, the tube is immediately turned over to allow the remaining gold slurry to smear to the side of the tube opposite where it originally settled. After about 2-5 minutes of air drying time, the piece of tubing is detached from the syringe and attached to a fluid outlet on the manifold. In various embodiments, the tube is connected to the manifold via the first intermediate tubing segment and a second intermediate tubing segment forming a Leur lock connection with the fluid outlet of the manifold. One in the art will appreciate that this configuration reduces the risk of cross-contamination between samples and/or samples and the manifold.

The luminal surfaces of pieces of tubing attached to the manifold are then dried by passing a gas, such as nitrogen, through the pieces of tubing. In various embodiments 0.1 to 0.2 LPM nitrogen gas is allowed to flow through the pieces of tubing. Monitoring of the drying process entails increasing or decreasing the nitrogen flow to allow the particles to dry without being blown out of the tube as well as optionally turning over the tube to distribute the particles on the interior of the tubing. For example, the pieces of tubing may be turned once or twice during the drying period. When particles such as gold are utilized, a color change from dark to light yellow is evident when the particles are completely dried. In some embodiments, the particles are allowed to dry for about 5-20 minutes. In some embodiments, the particles are completely dried in less than about 20, 15, 14, 13, 12, 11, 10, 9, 8, 7, 6, or 5 minutes.

The method does not require, and in exemplary embodiments does not include, continuous rotation of the pieces of tubing that include the coated particle suspension as it dries. For example, the method provided herein does not include attachment of a piece of tubing to a device that rotates the tubing for a period of time during which a gas such as nitrogen is passed through the tubing. Rather, the particle suspension dries within the pieces of tubing while gas is passed through the lumens of the pieces of tubing that may be turned over (rotated approximately 180 degrees) one, two, three, or more times during the drying period (for example, no more than ten times, no more than eight times, no more than six times, or no more than five times).

Once dried, each piece of tubing is cut into individual sample cartridges for use in a gene gun to transform cells. In particular embodiments, the tube is cut into segments of about 0.5 inches in length to fit the cartridge holder of the Bio-Rad® Helios® Gene Gun. Other lengths may be desired based on the gene gun device to be used.

Accordingly, in another aspect, the present invention provides a method of delivering a biological molecule or a nucleic acid molecule to a cell. The method includes providing a piece of tubing prepared according to the method of the invention, wherein the lumen of the piece of tubing includes deposited particles conjugated to a biological molecule, and contacting the cell with the particles deposited within the piece of tubing via a gene gun device. The method may be utilized in performing biological assays, such as gene delivery and cellular transformation.

In various embodiments, the simultaneous drying capability of the present invention reduces the time input to approximately two hours for twelve bullet sets. Additionally, the present invention allows for scale-down of the number of bullets when fewer than forty bullets per sample are desired as required using the Bio-Rad® Tubing Prep Station™. The Tubing Prep Station™ is designed to make 40 bullets per DNA sample, and reduction of this number is impractical because the apparatus is too long to be able to manipulate a shorter length of tubing. The steps taken during preparation of the gold suspension are the same in the present invention as they are for the Bio-Rad® Tubing Prep Station™, but all of the volumes are scaled down to approximately 25% of those recommended for the Bio-Rad® Tubing Prep Station™ in order to make 10 bullets per DNA sample. This reduces waste of materials when only a few bullets from each DNA sample are needed. In various examples, as few as 4, 5, or 6 bullets may be generated from a sample solution by scaling down the volumes proportionately, thereby providing for economies of scale.

A further aspect of the invention are particles for bombardment of cells coated with at least one RNA molecule and at least one DNA molecule. At least one RNA molecule can be a guide RNA, which can be, for example, a crRNA (that does not include a tracr sequence) or a chimeric guide RNA (also called a single guide or sg RNA) that includes the target sequence as well as the tracr sequence. In some embodiments, at least one DNA molecule can be a selectable marker cassette. In particular embodiments, a preparation of particles for bombardment of cells can be coated with a donor fragment that includes a selectable marker cassette and at least one guide RNA. In some embodiments, a preparation of particles for bombardment of cells can be coated with a donor fragment that includes a selectable marker cassette and at least two guide RNAs. Alternatively or in addition, a preparation of DNA and RNA coated particles for cell bombardment can include particules coated with at least two guide RNAs and at least two donor DNAs, each of which comprises a different selectable marker cassette conferring resistance to a different antibiotic. In such embodiments, transformants can be be selected for resistance to both markers to select for strains having the genome altered at sites targeted by both guide RNAs.

Also included herein are methods for transforming cells by particle bombardment in which the particles are coated with both RNA and DNA. The particles can be coated with at least one DNA molecule that comprises a selectable marker cassette and at least one guide RNA. In some embodiments of the method, the particles are coated with at least two guide RNAs. In some embodiments of the method, the particles are coated with at least two guide RNAs and at least two DNA molecules that each comprise a different selectable marker cassette.

A preparation of particles of particle bombardment of cells, wherein the particles are coated with at least one RNA molecule and at least one DNA molecule. The particles can be coated with at least one guide RNA or crRNA, and can optionally further be coated with a DNA molecule that comprises a selectable marker cassette. In some embodiments, the particles can be coated with at least two guide RNAs or crRNAs, and can optionally further be coated with a DNA molecule that comprises a selectable marker cassette.

### Examples

The following examples are provided to further illustrate the embodiments of the present invention, but are not intended to limit the scope of the invention. While they are typical of those that might be used, other procedures, methodologies, or techniques known to those skilled in the art may alternatively be used.

Parachlorella is a green alga in the Chlorophyte phylum. A strain of Parachlorella was isolated from the environment and given the designation WT-1185. Media used for the growth of Parachlorella included the following PM074 and PM126.

PM074 is a nitrogen replete (“nitrate-only”) medium that is 10×F/2 made by adding 1.3 ml PROLINE® F/2 Algae Feed Part A (Aquatic Eco-Systems) and 1.3 ml PROLINE® F/2 Algae Feed Part B (Aquatic Eco-Systems) to a final volume of 1 liter of a solution of Instant Ocean salts (35 g/L) (Aquatic Eco Systems, Apopka, Fla.). Proline A and Proline B together include 8.8 mM NaNO₃, 0.361 mM NaH₂PO₄.H₂O, 10×F/2 Trace metals, and 10×F/2 Vitamins (Guillard (1975) Culture of phytoplankton for feeding marine invertebrates. in “Culture of Marine Invertebrate Animals.” (eds: Smith W. L. and Chanley M. H.) Plenum Press, New York, USA. pp 26-60).

PM126 is a nitrogen replete (“nitrate-only”) medium that is 10×F/2 made by adding 1.3 ml PROLINE® F/2 Algae Feed Part A (Aquatic Eco-Systems) and 1.3 ml PROLINE® F/2 Algae Feed Part B (Aquatic Eco-Systems) to a final volume of 1 liter of a solution of Instant Ocean salts (7 g/L) (Aquatic Eco Systems, Apopka, Fla.). Proline A and Proline B together include 8.8 mM NaNO₃, 0.361 mM NaH₂PO₄.H₂O, 10×F/2 Trace metals, and 10×F/2 Vitamins (Guillard (1975) Culture of phytoplankton for feeding marine invertebrates. in “Culture of Marine Invertebrate Animals.” (eds: Smith W. L. and Chanley M. H.) Plenum Press, New York, USA. pp 26-60).

### Example 1

### Coated Particle and Sample Cartridge Preparation

To prepare DNA-coated gold bullets for use in Helios® Gene Gun (Bio-Rad®, Hercules, Calif.), DNA was precipitated onto gold particles and resuspended in 100% ethanol/10 μg/ml PVP solution as detailed in the Helios® manual and described in U.S. Pat. No. 8,883,993, incorporated herein by reference in its entirety. In addition, a second, modified method was developed that was similar to that described in the Helios® manual with the exception that 1) the volumes were calculated to make ten bullets instead of forty as described in the manual (see Table 1); and 2) the method did not use the Bio-Rad® Tubing Prep Station™ (Table 1) or a similar device in which both ends of a piece of tubing were fixed to a rotating apparatus. Instead, in the modified method a manifold device was employed in which bullets were dried to the internal surfaces of multiple pieces of tubing simultaneously, and the method did not include continuous rotation of the tubing.

To prepare the bullet cartridges (pieces of tubing having dried DNA-coated bullets on the internal surface) by the second method, while the DNA/gold suspension was being prepared by precipitation of the DNA onto the particles using spermidine and calcium chloride (see Example 3), one 7 inch length of Tefzel™ tubing for each DNA sample (four total, plus a no DNA control, see Table 2) was pre-dried by insertion into the flexible tubing attached to the manifold drier (shown in FIG. 2) and left for at least fifteen minutes with approximately 0.4 LPM nitrogen flowing through the pieces of tubing to eliminate environmental humidity accumulation inside of the Tefzel™ tubing.

After preparing the DNA/gold suspension and pre-drying the pieces of Tefzel™ tubing, each of the pieces of flexible tubing were disconnected from the manifold drier at the Leur locks and individually (one at a time) attached to a 10 mL syringe. The DNA/gold suspension of an individual preparation was mixed well and drawn into the piece of Tefzel™ tubing by application of suction by the syringe. While still connected to the syringe, the Tefzel™ tubing was laid on a flat surface for five minutes while the gold settled out of solution and adhered to the inside of the tubing. Since the percentage of the gold that adhered to the tubing was influenced by the settling time, it was important to keep the settling time consistent for every sample; the best practice was to use a timer and stagger the step of drawing the gold into the Tefzel™ tubing to allow for adequate handling time for each sample in subsequent steps.

After approximately five minutes of settling time, pressure was applied with the syringe to gently push the ethanol out of each piece of tubing. The tubing segments were then immediately turned over to allow the remaining gold slurry to smear to the side of the Tefzel™ tubing segment opposite where it originally settled. After 2-5 minutes of air drying time, each Tefzel™ tubing piece was detached from the syringe and moved back onto the manifold drier with 0.1-0.2 LPM nitrogen flowing through the manifold. (Monitoring of the drying process can entail increasing or decreasing the nitrogen flow to allow the gold to dry without being blown out of the Tefzel™ tubing, as well as occasionally turning over the Tefzel™ tubing to more evenly coat the interior of the tubing.) When the gold was completely dried as evidenced from a visible color change from dark to light yellow, each individual Tefzel™ tubing segment was removed from the flexible tubing and cut into half-inch pieces for use in the Helios® Gene Gun.

### Example 2

### Transformation of Cells

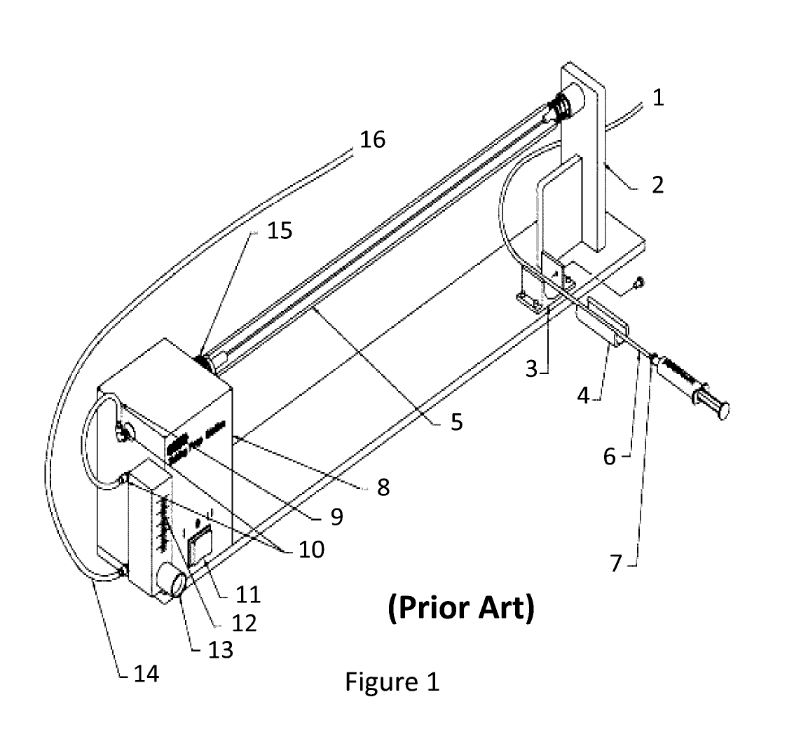

Transformation of Parachlorella WT1185 was accomplished using the Bio-Rad® Helios® Gene Gun System. The general protocol was developed using the manufacturer's instruction manual (Bio-Rad®, USA). DNA for transformation was precipitated onto gold particles and the gold particles were adhered to the inside of lengths of tubing using the Bio-Rad® Tubing Prep Station™ (FIG. 1) according to the manufacturer's protocol and as detailed in U.S. Pat. No. 8,883,993 (incorporated herein by reference in its entirety) or using the Manifold Drier (FIG. 2) as detailed above in Example 1. In each case, for transformation, a burst of helium gas was fired through the tubing cartridges by the Gene Gun which projected the DNA-coated gold particles into WT-01185 algal cells adhered on solid non-selective media. The following day, cells were moved onto selective media for growth of transformed colonies.

Quantities of materials used for each method are described in Table 1 below, which demonstrates a savings in all reagents used by employing the Manifold Drier methods where 10 or fewer bullet cartridges are required.

TABLE 1
Reagents and Materials required for Bullet Preparation
using Tubing Prep Station (40 Bullet cartridges) v.
Manifold Bullet Prep (10 Bullet cartridges)

| Material | 40-bullet | 10-bullet |
|---|---|---|
| Mg 0.6 μm gold (Bio-Rad ® #165-2262) | 20 | 5 |
| Inches Tefzel ™ Tubing (Bio-Rad ®) #165-2441) | 25 | 8 |
| μL 50 mM Spermidine | 100 | 50 |
| μg DNA | 40 | 2-10 * |
| μL 1M CaCl2 | 100 | 50 |
| mL 100% ethanol with 0.01 mg/mL PVP | 2.5 | 0.625 |

Two scale-up schemes were used for growth of cultures for transformation. In the first, a 200 mL seed culture inoculated to 3×10⁶ cells/mL three days before transformation was used to inoculate a 1 L culture to 3×10⁶ cells/mL one day before transformation. In the second, a 100 mL seed culture inoculated to 1×10⁶ cells/mL six days before transformation was used to inoculate a 1 L culture to 1×10⁶ cells/mL two days before transformation. Both versions have reliably resulted in mid-log culture at 1-3×10⁷ cells/mL for use on the day of transformation; the second version has been selected as the standard method. Cell counts were determined using a BD Accuri™ C6 Flow Cytometer. Cultures were grown in PM074 or PM126 media in a Conviron™ Incubator at 25° C. 1% CO₂ shaking at 130 rpm in a 16:8 light:dark cycle.

On the day of transformation, cell cultures were pelleted by centrifugation at 4500×g for twenty minutes. Cells were resuspended in 50 mL osmoticum (250 mM mannitol/250 mM sorbitol 0.1 μm filter-sterilized) and incubated for 1-2 hours at room temperature. The purpose of osmotic pre-treatment was to minimize the risk of cells bursting when struck by the microprojectiles by reducing the volume contained within the cell walls by placement in a hypertonic environment.

After osmotic pre-treatment cells were concentrated to 4×10⁹ cells/mL in osmoticum, and 50 μL of cell suspension was painted in each of five 4 cm-diameter circles on a 13 cm-diameter shooting plate containing 2% agar PM074 solid medium. When the cells were completely dried, the Helios® Gene Gun was used to fire two bullets per cell circle at 600 psi from a distance of 3-6 cm from the plate. In total for each individual DNA, 10 replicate bullets were fired at 1×10⁹ cells divided among 5 cell circles. Cells were left on the shooting plates overnight in ambient benchtop conditions.

The day after transformation, cells from replicate cell circles were pooled together by washing the bombarded plates with liquid PM074 or PM126 media. Recovered cells were plated onto selective media (PM074 containing zeocin 250 mg/L or PM126 containing 200 mg/L zeocin) at an intended density of 1×10⁹ cells per 22×22 cm agar plate.

Table 2 provides the results of the transformations using four different DNA preparations. In each case multiple transformants were obtained. Zeocin-resistant colonies appeared 10-14 days after bombardment. The use of the Manifold Drier produced gene bullet cartridges that were at least as effective as the gene bullet cartridges produced by the Bio-Rad® Tubing Prep Station™ in transforming cells while reducing reagents and allowing simultaneous prepartation of multiple sample bullets.

TABLE 2
Number of transformants per 10⁹ cells bombarded
using DNA-coated gold particles dried using the
Bio-Rad ® Tubing Prep Station or Manifold Drier.

| | Bio-Rad ® Tubing | | | |
|---|---|---|---|---|
| | Prep Station | | Manifold Drier | |
| | Experi- | Experi- | Experi- | Experi- |
| Ble-encoding DNA | ment 1 | ment 2 | ment 3 | ment 4 |
| pSGE-6530: excised Zeo^{R} | 59 | 41 | 100 | 32 |
| cassette (SEQ ID NO: 1) | | | | |
| pSGE030 linerarized vector | 27 | 91 | 70 | |
| pSGE-6543: excised Zeo^{R} | | 180 | | 544 |
| cassette (SEQ ID NO: 2) | | | | |
| pSGE043 linerarized vector | | 230 | 5376 | |
| No DNA | 0 | 0 | 0 | 0 |

### Example 3

### Preparation of Particles Coated with Both DNA and RNA Molecules

Chloroplastic SRP54 (cpSRP54) is a polypeptide that functions in the insertion of chlorophyll-ginding polypeptides into thylakoid membranes of the chloroplast. Reduction or elimination of the cpSRP54 polypeptide results in a reduced photosynthetic antenna and reduced chlorophyll content of affected cells; thus, knockout or knockdown of the cpSRP54 gene in algae provides a visible pale green phenotype (see U.S. Patent application publication US 2016/0304896, incorporated herein by reference in its entirety). The ability to knockout or knockdown genes using cas/CRISPR systems can be enhanced by cotransformation of guide RNAs and donor DNAs that include selectable markers, particularly in cells or microorganisms such as some algae that are difficult to transform by other means such as electroporation. Co-transformation of RNA and DNA into algal strain Parachlorella WT-01185 by particle bombardment was tested using the cpSRP54 gene as the gene target.

In a standard precipitation of DNA onto gold microprojectiles, such as that provided in the Helios® Gene Gun manual, gold powder is weighed into a 1.5 mL tube and suspended in spermidine. To the gold/spermidine slurry DNA and calcium chloride are added. After a ten-minute room temperature incubation, the sample is pelleted and washed three times with ethanol. The washed gold with adhered DNA is resuspended in an ethanol/PVP solution which is then applied to the Tefzel™ tubing (see also U.S. Pat. No. 8,883,993, incorporated herein by reference in its entirety).

In the RNA bullet preparation method described in the Helios® manual, the measured gold powder is suspended first in aqueous RNA Ammonium acetate (one tenth volume) and two volumes of isopropanol are added, and the sample is incubated at −20° C. for 1 hour to precipitate the RNA in the presence of the gold. After the 1 hour incubation, the sample is pelleted, washed, and applied to Tefzel™ tubing in the same way as DNA coated bullets.

To coat particles for bombardment transformation of cells with both DNA and RNA, in a first method, the RNA protocol described in the Helios® manual (bio-rad.com/en-us/product/helios-gene-gun-system, document M1652411) and provided in Example 1, above, was followed but with simultaneous addition of RNA and DNA. Specifically, a 30 μL volume containing 5 μg Zeocin-resistance encoding DNA pSG-6543 digested with AscI/NotI-HF (SEQ ID NO:2) and 20 μg of a guide RNA targeting SRP54 (SEQ ID NO:3), prepared using DNA oligomers that incorporated a T7 promoter (SEQ ID NO:4 and SEQ ID NO:5) as described in U.S. Patent application publication US 2016/0304896, incorporated herein by reference in its entirety, were added to 5 mg gold particles and vortexed. To the resulting slurry, 3 μL 5M NH₄OAc and 66 μL isopropanol were added, and the sample was incubated at −20° C. overnight. The following day, the gold was washed and applied to Tefzel™ tubing as described in Example 1, above.

In a second method for coating particles with both RNA and DNA, the DNA was coated on gold particles using the standard DNA protocol (also provided in the Helios® manual, bio-rad.com/en-us/product/helios-gene-gun-system, document M1652411), and then the guide RNA was precipitated in the presence of the DNA-coated gold using the standard RNA protocol. In this method, a slurry was made from 5 mg 0.6 μm gold particles in 50 μL 50 mM spermidine, to which 5 μg Asc/Not digested pSGE06543 (SEQ ID NO:2) was added. Calcium chloride (50 μL of a 1M CaCl₂ solution) was then added drop-wise while vortexing. After 10 minutes' incubation at room temperature, the DNA-coated gold was pelleted and resuspended in 30 μL containing 20 μg of SRP54 guide RNA (SEQ ID NO:3). To this slurry, 3 μL 5M NH₄OAc and 66 μL isopropanol were added, and the sample was then incubated at −20° C. overnight. The following day, the gold was washed and applied to Tefzel™ tubing according to the standard protocol.

Bullets for a minus-guide control were prepared using the RNA protocol from Method 1 with the exception that no guide RNA was added.

Cas9-expressing Parachlorella strain GE-15699 (see U.S. Patent application publication US 2016/0304896, incorporated by reference in its entirety) was cultured, Helios® bombarded with the DNA/RNA bullets, and plated on PM074 agar medium that included 250 mg/mL zeocin (U.S. Patent application publication US 2016/0304896). After three weeks' growth, 12 colonies with the desired pale phenotype (indicative of attenuated expression of the SRP54 gene) were suspended in 50 μL PM074 media, and 5 μL cell suspensions were spotted onto both PM074/zeo250 agar and PM074 agar without antibiotic alongside the GE-15699 Cas9-expressing control, and the Parachlorella SRP54 classical mutant NE-7557 (US 2016/0304896, incorporated by reference).

After the spotted cells were grown to maturity, colony PCR was performed using biomass from the PM074/zeo250 plate. Scant loopfuls of cells were boiled 99° C. for 15 minutes in 80 μL 5% Chelex. The boiled suspensions were centrifuged briefly, and 2 μL of the supernatants were used as templates for colony PCR genotyping to confirm disruption of the SRP54 coding region using the primers of SEQ ID NO:6 and SEQ ID NO:7.

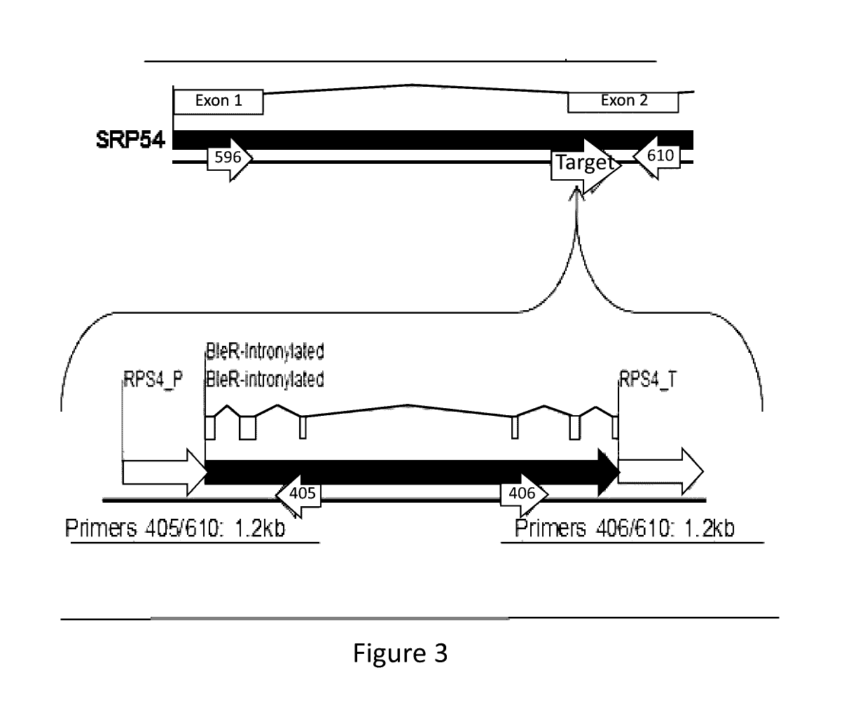

Because the pSGE-6543 ble cassette was larger than what could be reliably PCR amplified, the genotyping strategy employed three primer pairs to screen for not only loss of the wild type SRP54 gene band (which occurs as a consequence of ble cassette integration in the target SRP54 gene locus) but also for presence of a junction fragment between the ble cassette and the target locus by pairing one ble-specific primer and one locus-specific primer in the diagnostic PCRs. Since the ble cassette can be integrated in multiple orientations, the junction amplification used the same ble primer paired with either the upstream or downstream locus primer. A knock-out line was defined as having either a) a larger-than-wild type locus-to-locus amplicon (generated by primer AE596 (SEQ ID NO:6) and primer 610 (SEQ ID NO:7)) or b) both a ble-to-locus amplicon in either or both orientations (generated by primer AE405 (SEQ ID NO:8) and primer 610 (SEQ ID NO:7) or generated by primer AE406 (SEQ ID NO:9) and primer 610 (SEQ ID NO:7)) and the loss of the wild type amplicon. This is shown schematically in FIG. 3. Diagnostic primer sequences are provided in Table 3.

TABLE 3
Primer sequences for diagnosing Cas9-mediated integration of
DNA fragment into SRP54 locus targeted by guide RNA.

| | |
|---|---|
| AE596: SRP54 upstream locus | TGCGACATGCAGCTTACTAACCTGCTCGACAT |
| | (SEQ ID NO: 6) |
| AE610: SRP54R downstream locus | CCCCCAGCCTCACATCCGCCTCAA |
| | (SEQ ID NO: 7) |
| AE405: Ble R internal | ACCCAAACCCATGCCAGTGTA |
| | (SEQ ID NO: 8) |
| AE406: Ble F internal | ACTGTATGCAGAGTGGTCTGAAGTG |
| | (SEQ ID NO: 9) |

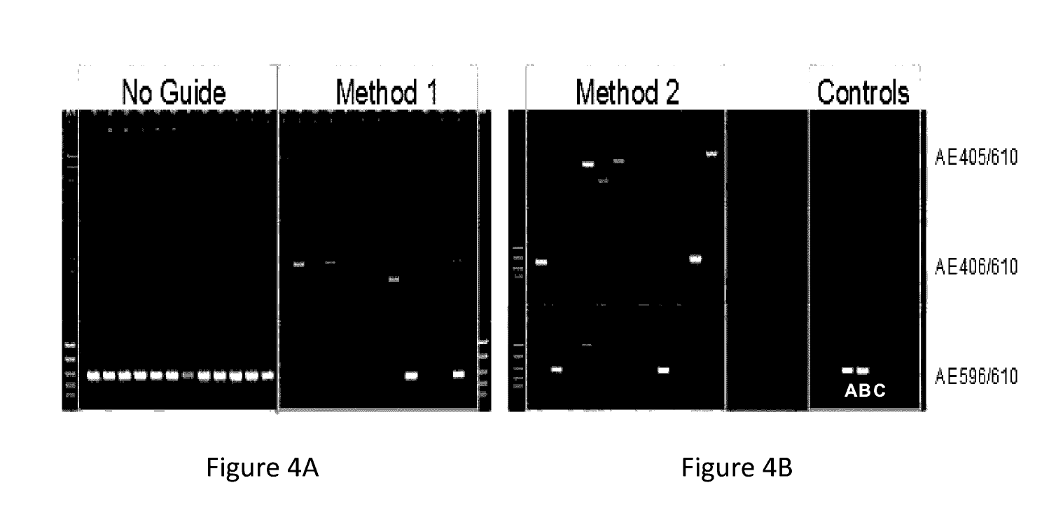

PCR results (FIGS. 4A and 4B) and observable phenotypes (lack of pale colonies) indicate that zero SRP54 knock-outs were generated from the no guide control. Conservatively, 3 out of 12 colonies from Method 1 (ethanol co-precipitation of DNA and RNA with particles) (FIG. 4A) and 6 out of 12 colonies from Method 2 (sequential coating of particles with DNA in spermidine followed by alcohol precipitation of RNA and particles) (FIG. 4B) had clear PCR evidence of integration of the ble cassette at the CRISPR locus and loss of the wild-type locus-to-locus amplicon. The pale phenotypes of the colonies confirm the SRP54 gene knock-out.

Although the invention has been described with reference to the above examples, it will be understood that modifications and variations are encompassed within the spirit and scope of the invention. Accordingly, the invention is limited only by the following claims.

SEQUENCES

SEQ ID NO: 1
DNA
Synthetic
Artificial
Zeocin resistance cassette from pS GE-6530
GGCCGCCCACCATGGGGGAGGTTTGAAGTGTGCGCCTGATATAATCATAC
ACCTAAAAGCACCACTTGCTGATTGTGAAGGGACTATGTCGTTTATGACG
GGACGTTACGCTGGCCGATGGTTTGAATTTGGACGCTGTGGTAGAATGTT
ATATGGACGTAAAGGTTGGCATATTGAAAATCGTCTTCACAGGCAAACTT
CTAGACGTGTGACCCACCGGTAAAACGACAAGCGTGGCGCGTCGATTGCG
CTTTGAACGTCGTTTGTTGGACTCCAGATGAACCTCAAAATCAAAGCGGT
GATTGACGAAAATCAAATGACAGCCCGCAAAATTTCATCAGCCTTCGGAT
CGGATTCTCAGAATCTGATTGTCCCTGCTGGCTACATTTATGAAATTTCG
TACATTTTGGCAGAAATGTCCCAATACCATAGCACTGCCGCCTGAGCTCA
CCCGAGCAATGCATACTGGGTACCTCGCCCATCTCGCCCTCTTTCCAAGC
CCAGTGCTGTTGTAAATAGCCAAAGGGCTCAGTAACAATGGCCAAACTGA
CATCCGCTGTTCCTGTGTTGACAGCAAGAGATGTTGCAGGTGCAGTGGAG
TTTTGGACAGATAGACTGGGGTTTAGCAGGGACTTTGTGGAGGACGATTT
TGCAGGAGTGGTGAGGGATGATGTGACACTGTTTATCTCAGCAGTGCAGG
ATCAAGTGGTGCCCGATAATACACTGGCATGGGTTTGGGTGAGAGGATTG
GATGAACTGTATGCAGAGTGGTCTGAAGTGGTGAGCACCAACTTTAGGGA
TGCAAGCGGACCTGCAATGACAGAGATTGGAGAACAACCTTGGGGAAGGG
AGTTTGCATTGAGAGATCCTGCAGGGAATTGCGTGCACTTTGTTGCAGAA
GAACAGGACTGAGCATAGCATCAGCCTGTGGCAGGGTTGTGGTAGGGCTG
AGTGGCAGGGTTAAAGGGGTTGCCTACCCCACCCCTACTCTCATGACACC
AGCAACAGCAGCAGCTCATGCAGTACTCAAATCACTGATGTCAATGGTGT
GACACATTTGGTTAAGGCTGCTTTTTAAAGTGCTGCTTTGGGGGCAGTGA
CTGTGCAGAGCTTGGAGCGTATCCCCATGTAATCAGAACCGACGAGAGTT
CGGGGCAACCTTTCATCTTCACATTTTTTGTGATCAGCTACAGAGTCTGA
AATCAAATAGAGGCTGCCATCTAAACGCAGGAGTCACAACGAAGGCGAAA
ACTCCAATTGCTGTACTCAATGCACTAAGTGATTGTTCAATGGATAAATA
CACTATGCTCAATTCATGCCAGCAGAGCTGCTCCTTCCAGCCAGCTACAA
TGGCTTTTTCCACGCCTTTTGAAGTATGAATGTTCAGCTTGCTGTGCTTG
ATGCATCACCATAAACACAATTCTACAACATTTCATGCCAACAACAGTAC
GGGCTTTCGG
SEQ ID NO: 2
DNA
Synthetic
Artificial
Zeocin resistance cassette from pS GE-6543
GGCCGCCCACCATGGGGGAGGTTTGAAGTGTGCGCCTGATATAATCATAC
ACCTAAAAGCACCACTTGCTGATTGTGAAGGGACTATGTCGTTTATGACG
GGACGTTACGCTGGCCGATGGTTTGAATTTGGACGCTGTGGTAGAATGTT
ATATGGACGTAAAGGTTGGCATATTGAAAATCGTCTTCACAGGCAAACTT
CTAGACGTGTGACCCACCGGTAAAACGACAAGCGTGGCGCGTCGATTGCG
CTTTGAACGTCGTTTGTTGGACTCCAGATGAACCTCAAAATCAAAGCGGT
GATTGACGAAAATCAAATGACAGCCCGCAAAATTTCATCAGCCTTCGGAT
CGGATTCTCAGAATCTGATTGTCCCTGCTGGCTACATTTATGAAATTTCG
TACATTTTGGCAGAAATGTCCCAATACCATAGCACTGCCGCCTGAGCTCA
CCCGAGCAATGCATACTGGGTACCTCGCCCATCTCGCCCTCTTTCCAAGC
CCAGTGCTGTTGTAAATAGCCAAAGGGCTCAGTAACAATGGCCAAACTGA
CATCCGCTGTTCCTGTGTTGACAGCAAGAGATGTTGCAGGTGCAGTGGAG
TTTTGTGAGTTCTGAGAAGCTGATTGTTGTTTAACTTCTTTGAAAGCTTT
ATCGAAGATTCTGCAAGCGATGAACATTGCTTGTCAAGACCGAGAGCTGC
ATGCCCACTTGACATCCAGCTTTGAACGGCTCTTCATGTTTGATTTGTTT
CTGATTGTAGGGACAGATAGACTGGGGTTTAGCAGGGACTTTGTGGAGGA
CGATTTTGCAGGAGTGGTGAGGGATGATGTGACACTGTTTATCTCAGCAG
TGCAGGATCAAGTGAGTGCAGCGTCAGCTGTGGCAGTTGTTGGCTTTCGT
CTCAGTCAGTAGTTTGCTGGGATTGATTATGGAGGGCACAGTTGCAATTT
TGAGTTGCACGTTGCGACAAGCGTGTTGACAAAGCGTGGTCAAGCCGGCC
AGTCTTGCCGGTGGCGGGTGGCTTGGTCTAACTTCCGCTCTACAGCAATC
GTTTTGTTCATGGTTACGGGGCTGGCGTGCCAGAAAGTCCTGGTCAGCCA
CCCTCGCTTCAAAGCCGTAGCCCAACAACTTTGCGAATATGTTCGATTTG
CAGGTGGTGCCCGATAATACACTGGCATGGGTTTGGGTGAGAGGTACAGC
TCTGCGTGCAACAGGTTGCAAGATGCAGCGCAGGTCTTCCCTGGTCAAAC
GATGTATGCAGAGTTGAGAGGCACTTGAGCTGGGTGAATGGCGTGGGCTC
GTAGGTAGTGTGCAGGGCAGGAAGGGCAGCCAATTTTGGAGTTGTGGTCC
GGTGTCGTTGCTTCGAGCCTTATTAGGACTCTTGCTCATCAAAGCGTTAG
TTGTGAATAAGTTGATCTGAAAGGATGTTATGTACAGCAAGCAGCAGCAG
TTAAGAGTCTGGGGAGTAGCTGCACAGGGCGAGGTGTCAAGATGGGAAGG
GTCCTGCCTCCTTATGTGTTTTTCCCTGTAGGGGAGGAAGCCTCTTATGG
GCAATGGTTGGGCATATTTTCCAGCCAGCCCTTCTTTCTATAGGGGCCAG
GGTGGGCCCAGCTCGTCTTGGCTTCCACCACCAGGAGAGTGAGGGCATTG
AAGGGCCATAAATAGTCCTCCCATCTACGTGCACCAGAGGGTGTCGTCTA
GGCTGTGCATGCCACGAGGGGAAGGAGCCAAGAATGAGTGTATGGGTTGT
TTTCATGTTTAGGCTGGGATAAAACTGTTTTCAATTGCGCCTGCCGGGTG
AAAACCACAGCAGCATCAGCAAGCTTGGAGAAGGCCAGCCCGCCCAGCAC
AGGCTCACGTTCCCACTCAGGCGGTCAGTCGGGCGGGGGTGTGAGTCAGG
CAGGCGAGGGTGTCTGTGCCTGACATCAGCACCTCTGCTTAGCCACTGCA
GCCCCTGGAGCAGGGTAGGGCGTCATTTGCAGCAATCACCTGCTGCCTCA
CACGTCGCAGCTTGGAATTTCAACGACCATCAGCGCTGGGGTTGTTGAGG
GATCATAGCAGATTTTGGTGCAGCCTGGTTGTCATGCTCTTTGTGGAATG
GCCTCTATGTTCGAGCAATTCGTTGGATGTTGAGGTGCTTGGGGACAGAG
AGTCGAATGATGGGCCAGGGTCAAACATGCGAGCGTTTGGCTGAGTCAGC
GGTTTTTGCTGGTCACTTTTTCTTTTGTTTCTTATTTAGGTTTGATGGAT
GTGTTTTGTGCTGCTGCCCTGAAGCTGCAGCAGCGTGTCTGCCCTGCGCT
ACTGCGGGCACCAAGGCTATGTGCTGGTGCACTCGGCTGCGCTGCACCTG
TGCACCTCGCACTCCGTCCAGCCTCCATGCAGCACACGTACTCACGGTGT
CCTCCTGACCTGTCGTACGCTATTCCAAACTTGCTCTTTTGCTGCCGCTG
CTCTCGTACACAATTGCTGTTGATTATCGATATCTAATCGAGCGCCTGCT
GACTGAACTCCGCAGGtTTGGATGAACTGTATGCAGAGTGGTCTGAAGTG
GTGAGCACCAACTTTAGGTGGGTGGGCTCTGAAGGAGGAGGAGGGAGCGG
GTGATTAAACAGGGCCTGCATGAAGAGGAGCAGGGGCTGCATGGACAGCA
GGGGGAAGGTGCAGAAGGGAGGGTCAAGCGGGGTTCAGGTGGCTGTGGGT
TTCTGCACGAGCAGTGAAAGAAGCTGTATCCTTCCACCTGCTTTCACTGG
CGAAAGGTTGAAAACAGGATGTCGCAGCTGGAAAGATGTTGCGCTGTCAA
GTGCAAGCCATGGTTGAGGGTATGCCTGTGTGCATGTGCTTCTTAAAGTT
ACTCCTGTTCTATGGTTCTGGGTGCTTGTTGTTTGTGGTGCAGGGATGCA
AGCGGACCTGCAATGACAGAGATTGGAGAACAACCTTGGGGAAGGGAGTT
TGCATTGAGAGATCCTGCAGGTGAGGGGGCATGTAAGCAATGGCAGGCAA
TTCAAGAACGAATCATTGCTGCAAATGCTGGGATGGTATGCAGCTGAGGT
ATCTATTGCCTTGTATTTTGTCTCGCATTGCATCGGTGGTGCGTTCTGTG
GCCTGAGGCACAGTTCTTGCTGTTTGATAAGGGTTCGACTGAGTTGTCGT
GTGTGCTGTGCTGCAGGcAATTGCGTGCACTTTGTTGCAGAAGAACAGGA
CTGAGCATAGCATCAGCCTGTGGCAGGGTTGTGGTAGGGCTGAGTGGCAG
GGTTAAAGGGGTTGCCTACCCCACCCCTACTCTCATGACACCAGCAACAG
CAGCAGCTCATGCAGTACTCAAATCACTGATGTCAATGGTGTGACACATT
TGGTTAAGGCTGCTTTTTAAAGTGCTGCTTTGGGGGCAGTGACTGTGCAG
AGCTTGGAGCGTATCCCCATGTAATCAGAACCGACGAGAGTTCGGGGCAA
CCTTTCATCTTCACATTTTTTGTGATCAGCTACAGAGTCTGAAATCAAAT
AGAGGCTGCCATCTAAACGCAGGAGTCACAACGAAGGCGAAAACTCCAAT
TGCTGTACTCAATGCACTAAGTGATTGTTCAATGGATAAATACACTATGC
TCAATTCATGCCAGCAGAGCTGCTCCTTCCAGCCAGCTACAATGGCTTTT
TCCACGCCTTTTGAAGTATGAATGTTCAGCTTGCTGTGCTTGATGCATCA
CCATAAACACAATTCTACAACATTTCATGCCAACAACAGTACGGGCTTTC
GG
SEQ ID NO: 3
RNA
Synthetic
Guide RNA targeting Parachlorella cpSRP54
GGGACAUGGUGCGCAAGGACGUUUUAGAGCUAGAAAUAGCAAGUUAAAAU
AAGGCUAGUCCGUUAUCAACUUGAAAAAGUGGCACCGAGUCGGUGCUUUU
UUU
SEQ ID NO: 4
DNA
Synthetic
Sense oligomer for producing guide RNA of
SEQ ID NO: 3
TAATACGACTCACTATAGGGACATGGTGCGCAAGGACGTTTTAGAGCTAG
AAATAGCAAGTTAAAATAAGGCTAGTCCGTTATCAACTTGAAAAAGTGGC
ACCGAGTCGGTGCTTTTTTT
SEQ ID NO: 5
DNA
Synthetic
Antisense oligomer for producing guide RNA of
SEQ ID NO: 3
AAAAAAAGCACCGACTCGGTGCCACTTTTTCAAGTTGATAACGGACTAGC
CTTATTTTAACTTGCTATTTCTAGCTCTAAAACGTCCTTGCGCACCATGT
CCCTATAGTGAGTCGTATTA
SEQ ID NO: 6
DNA
Synthetic
SRP54 upstream Primer AE596
TGCGACATGCAGCTTACTAACCTGCTCGACAT
SEQ ID NO: 7
DNA
Synthetic
SRP54 downstream Primer AE610
CCCCCAGCCTCACATCCGCCTCAA
SEQ ID NO: 8
DNA
Synthetic
Ble resistance gene internal forward primer AE405
ACCCAAACCCATGCCAGTGTA
SEQ ID NO: 9
DNA
Synthetic
Ble resistance gene internal reverse primer AE406
ACTGTATGCAGAGTGGTCTGAAGTG

What is claimed is:

1. A method for simultaneously depositing particles on the interior surfaces of a plurality of separate pieces of tubing, the method comprising:
a) preparing a suspension of particles coated with a biological substance in an evaporable liquid;
b) introducing the particle suspension into each separate piece of tubing of the plurality of tubing pieces; and
c) simultaneously passing a gas through each piece of tubing via a manifold to dry the particles onto the interior surface if each of the tubing pieces, the manifold comprising: i) an elongated lumen defining a central fluid flow pathway; and ii) a plurality of fluid connector ports disposed along the lumen, wherein each piece of tubing is in fluid connection with a fluid connector port whereby the gas is allowed to simultaneously flow from the lumen into each piece of tubing, thereby simultaneously depositing particles on the interior surface of each of the pieces of tubing.

2. The method of claim 1, wherein (b) comprises:
coupling the syringe to an end of a piece of tubing; and
drawing the suspension of particles into the piece of tubing using the syringe.

3. The method of claim 1, wherein a different suspension of particles is introduced into two or more separate pieces of tubing.

4. The method of claim 3, wherein the particles of each different suspension are coated with a different biological substance.

5. The method of claim 4, wherein the biological substance comprises one or more nucleic acid molecules.

6. The method of claim 5, wherein the biological substance comprises at least one DNA molecule and at least one RNA molecule.

7. The method of claim 1, further comprising rotating the tube about its longitudinal axis to distribute the particles on the interior surface of the tubing.

8. The method of claim 1, with the proviso that the method does not include continuous rotation of each tube while (b), (c) or both (b) and (c) are performed.

9. The method of claim 1, wherein each fluid connector port optionally comprises a valve to regulate gas flow into each piece of tubing.

10. The method of claim 1, wherein each piece of tubing is less than about 12, 11, 10, 9, 8, 7, 6, 5 or 4 inches in length.

11. The method of claim 1, wherein each piece of tubing is about 5-7 inches in length.

12. The method of claim 1, wherein at least 2, 3, 4, 5, 6, 7, 8, 9 10, 11, 12 or more separate pieces of tubing are utilized.

13. The method of claim 12, wherein each separate piece of tubing comprises a different suspension of particles, each different suspension comprising a different biological substance.

14. An apparatus comprising:
a) a manifold comprising: i) an elongated lumen defining a central fluid flow pathway; and ii) a plurality of fluid connector ports disposed along the lumen; and
b) two or more pieces of tubing,
wherein each of the two or more pieces of tubing is in fluid connection with a fluid connector port of the manifold.

15. The apparatus of claim 14, wherein the interior surface of at least one piece of tubing of the two or more pieces of tubing is deposited with a particle coated with a different biological substance than coats a particle deposited in at least one other piece of tubing of the two or more pieces of tubing.

16. A method of coating particles with at least one DNA molecule and at least one RNA molecule, comprising:
providing a slurry of metal particles in a solution of spermidine and at least one DNA molecule;
adding calcium chloride to the slurry of metal particles;
pelleting the metal particles;
resuspending the particles in aqueous solution;
adding RNA to the particles in aqueous solution;
alcohol precipitating the RNA and metal particles; and
resuspending the particles in alcohol to provide particles coated with DNA and RNA.

17. The method of claim 16, wherein the particles are gold or tungsten.

18. The method of claim 16 wherein alcohol precipitating is precipitating with ethanol or isopropanol.

19. A preparation of particles of particle bombardment of cells, wherein the particles are coated with at least one RNA molecule and at least one DNA molecule.

20. A preparation of particles of particle bombardment of cells, wherein the particles are coated with at least two RNA molecules and at least one DNA molecule.
